(19) United States
(12) Patent Application Publication
(10) Pub. No.: US 2022/0074889 A1
(43) Pub. Date: Mar. 10, 2022

(54) Device and Method for Biomolecule Measurement

(71) Applicant: Nippon Telegraph and Telephone Corporation, Tokyo (JP)

(72) Inventors: Suzuyo Inoue, Tokyo (JP); Katsuyoshi Hayashi, Tokyo (US); Michiko Seyama, Tokyo (JP)

(21) Appl. No.: 17/415,889

(22) PCT Filed: Dec. 4, 2019
(86) PCT No.: PCT/JP2019/047439

§ 371 Date: Jun. 18, 2021

(30) Foreign Application Priority Data
Dec. 19, 2018 (JP) 2018-237028

(51) Int. Cl. G01N 27/416; G01N 21/41; G01N 27/327

(52) U.S. Cl. CPC G01N 27/416; G01N 27/327; G01N 21/41

(57) ABSTRACT

Particles of a sustained-release gel which is converted into a sol by reacting with a product produced by a reaction of biomolecules with an enzyme, are disposed on a measurement unit, and a measurement target biomolecule is brought into contact with the particles. Due to this contact, a product is produced according to a reaction of the biomolecule with the enzyme contained in the particles. According to the reaction with the produced product, the sustained-release is converted into a sol, and a plurality of contained detection molecules are released to the outside of the particles.

### CROSS-REFERENCE TO RELATED APPLICATIONS

This application is a national phase entry of PCT Application No. PCT/JP2019/047439, filed on Dec. 4, 2019, which claims priority to Japanese Application No. 2018-237028, filed on Dec. 19, 2018, which applications are hereby incorporated herein by reference.

### TECHNICAL FIELD

The present invention relates to a biomolecule measurement device and method for measuring biomolecules.

### BACKGROUND

The shape and abundance of biological markers (biochemical substances) present in biological samples such as blood and saliva change in response to abnormalities that occur in living organisms. At an early stage in which abnormalities occur in living organisms, if changes in biochemical substances corresponding to the abnormalities that have occurred can be detected, it can be expected that earlier treatment, in a state transition state without subjective symptoms, would be able to be performed and treatment completed in a shorter time. Therefore, if the above changes in biological markers are detected, it is possible to reduce the physical and mental burden on patients themselves and medical expenses.

In recent years, in view of such a background, various measurement technologies for medical applications have been researched and developed in order to accurately detect biochemical substances. In order to detect a specific biochemical substance in a sample solution, a method in which functional biomolecules or compounds having molecular selectivity corresponding to specific chemical molecules are fixed to a surface of a substrate in advance, and the sample solution is caused to flow thereon, and thus the specific biochemical substance in the sample solution is bonded to functional biomolecules, and this bonding is electrochemically or optically detected is generally used (refer to NPL 1).

A measurement chip to which functional biomolecules are fixed, which is produced by a conventional fixing method, is stored in a dry state or packed and stored in a rigid pouch together with a buffer solution in order to prevent drying.

On the other hand, regarding the social background for biosensors, in response to the social background of aging and diversification of lifestyles, research and development of inspection technologies (systems) that will allow clinics and pharmacies and individuals in the future to easily perform pathological examinations that are currently performed only at specific medical institutions have been conducted. In order to easily perform living organism inspection, a technology for performing measurement from a small amount (>10 μL) of a sample obtained without invasion, using a small detection device, and without operations by an operator is required.

Regarding such a small detection device, there is an electrochemical sensor that measures a biochemical substance using an electrochemical reaction. Since this electrochemical sensor can detect a small amount of current, it is suitable in principle for detecting a small amount of biochemical substances that cause a redox reaction. The inventors have realized a sensor system that measures a biochemical substance using an enzymatic reaction and a redox membrane using a flow cell (refer to PTL 2). Electrodes can be microminiaturized by process manufacturing and thus are expected to be used for on-site biosensor technology.

In addition, in refractive index measurement (SPR measurement) using surface plasmons, a specific signal can be obtained simply by a direct bond between functional biomolecules and biochemical substances in the sample without requiring labeling with molecules that cause fluorescence or luminescence (for example, refer to PTL 3 and PTL 4). In this technology, an opening through which a sample solution is introduced is formed on a substrate, a metal thin membrane is formed inside the opening, and fine molecules are fixed to the metal thin membrane to form a biochip. The inventors have already realized an automatic introduction mechanism for a liquid sample that can be applied to a disposable chip (refer to NPL 2).

In the SPR measurement, for example, in the case of an antigen-antibody reaction, it is possible to measure an adsorption rate at which antigens (biochemical substances) and antibodies (functional biomolecules) bind, antigens are quantified in a short time in units of minutes and the measurement is completed. Therefore, since it is possible to realize a disposable chip with which measurement is possible in a short time, it is anticipated it will be used for a biochip technology that can be used in the field in which inspection is performed.

### CITATION LIST

### Patent Literature

PTL 1 Japanese Patent Application Publication No. 2001-061497

PTL 2 Japanese Patent Application Publication No. 2005-024456

PTL 3 Japanese Patent Application Publication No. 2010-008361

### Non Patent Literature

NPL 1 X. D. Hoa et al., “Towards integrated and sensitive surface plasmon resonance biosensors: A review of recent progress”, Biosensors and Bioelectronics, vol. 23, pp. 151-160, 2007.

NPL 2 T. Horiuchi et al., “Passive Fluidic Chip Composed of Integrated Vertical Capillary Tubes Developed for On-Site SPR Immunoassay Analysis Targeting Real Samples”, Sensors, vol. 12, pp. 7095-7108, 2012.

NPL 3 M. Ikeda et al., “Installing logic-gate responses to a variety of biological substances in supramolecular hydrogel-enzyme hybrids”, Nature Chemistry, vol. 6, pp. 511-518, 2014.

NPL 4 S. Takeuchi et al., “An Axisymmetric Flow-Focusing Microfluidic Device”, Advanced Materials, vol. 17, no. 8, pp. 1067-1072, 2005.

### SUMMARY

### Technical Problem

As described above, it is desirable for biosensors to be able to perform measurement anytime and anywhere for applications. However, in the current technology, functional biomolecules such as antibodies and enzymes are fixed by coating during measurement chip production, and in order to maintain activities thereof, it is essential to store them in a dark room under a low temperature condition. In addition, since there is a time limit to an activation time during which stability of biofunctional molecules fixed to the measurement chip is secured, the measurement chip has an expiration date.

In addition, since the measurement chip is basically disposable, it is desirable for the quantitative accuracy of the functional biomolecules fixed to the measurement chip to be uniform in order to use them without error between measurement chips. In addition, since determined target molecules are fixed to the measurement chip, it is necessary to prepare a dedicated measurement chip according to the target.

In addition, in order to efficiently perform measurement in a short time, it is desirable for the measurement target biochemical substance to be quantified without any chemical modification and with a small number of measurement processes. However, in order to improve the detection sensitivity, generally, a detection target biochemical substance is chemically modified with fluorescence. Therefore, when a supply system of a fluorescent component for chemical modification is provided in addition to a measurement target supply system and a functional biomolecule supply system, the device is complicated, and it is difficult to reduce the size of the device itself. Also, if reduction of the size is attempted using a micro flow path, there are problems that the number of measurement processes increases and the flow path structure becomes complicated.

Embodiments of the present invention have been made in order to address the above problems, and an objective of the present invention is to enable biomolecules to be efficiently measured in a short time with high detection sensitivity without using a dedicated measurement chip having an expiration date.

### Means for Solving the Problem

A biomolecule measurement device according to embodiments of the present invention includes a measurement device having a measurement region in which detection molecules are measured; and particles of a sustained-release gel containing an enzyme for measurement of target biomolecules and a plurality of the detection molecules, the sustained-release gel configured to be converted into a sol by reacting with a product produced by a reaction of the biomolecules with the enzyme and release the detection molecules.

In one configuration example of the biomolecule measurement device, the detection molecules are redox molecules, and the measurement device includes a first electrode and a second electrode disposed in the measurement region, and measures the detection molecules according to an electrochemical reaction.

In one configuration example of the biomolecule measurement device, the detection molecules have a smaller molecular weight than the biomolecules, and the measurement device measures the detection molecules by a surface plasmon resonance method.

The biomolecule measurement device according to embodiments of the present invention includes a membrane of a sustained-release gel containing an enzyme for measurement of target biomolecules and a plurality of the detection molecules, the sustained-release gel configured to be converted into a sol by reacting with a product produced by a reaction of the biomolecules with the enzyme and release the detection molecules; and a measurement device for measuring the change in the thickness of the membrane by a surface plasmon resonance method, wherein the detection molecules have a larger molecular weight than the biomolecules.

A biomolecule measurement method according to embodiments of the present invention includes a first process in which particles of a sustained-release gel are prepared, the sustained-release gel containing an enzyme for measurement of target biomolecules and a plurality of the detection molecules, and configured to be converted into a sol by reacting with a product produced by a reaction of the biomolecules with the enzyme and release the detection molecules; a second process in which the biomolecules are brought into contact with the particles; and a third process in which, after the biomolecules are brought into contact with the particles, the detection molecules are measured.

In one configuration example of the biomolecule measurement method, the detection molecules are redox molecules, and in the third process, the detection molecules are measured according to an electrochemical reaction.

In one configuration example of the biomolecule measurement method, the detection molecules have a smaller molecular weight than the biomolecules, and in the third process, the detection molecules are measured by a surface plasmon resonance method.

A biomolecule measurement method according to embodiments of the present invention includes a first process in which a membrane of a sustained-release gel is prepared, the sustained-release gel containing an enzyme for measurement of target biomolecules and a plurality of the detection molecules, and configured to be converted into a sol by reacting with a product produced by a reaction of the biomolecules with the enzyme and release the detection molecules; a second process in which the biomolecules are brought into contact with the membrane; and a third process in which, after the biomolecules are brought into contact with the membrane, the change in the thickness of the membrane is measured by a surface plasmon resonance method, wherein the detection molecules have a larger molecular weight than the biomolecules.

### Effects of Embodiments of the Invention

As described above, according to embodiments of the present invention, since detection molecules measured by the measurement device and enzymes for measurement of target biomolecules are contained in a sustained-release gel which is configured to be converted into a sol by reacting with a product produced by a reaction of the biomolecules with the enzyme, it is possible to obtain an excellent effect of efficiently measuring biomolecules in a short time with high detection sensitivity without using a dedicated measurement chip having an expiration date.

### BRIEF DESCRIPTION OF THE DRAWINGS

FIG. 1A is an explanatory diagram illustrating a biomolecule measurement method according to Embodiment 1 of the present invention.

FIG. 1B is an explanatory diagram illustrating a biomolecule measurement method according to Embodiment 1 of the present invention.

FIG. 1C is an explanatory diagram illustrating a biomolecule measurement method according to Embodiment 1 of the present invention.

FIG. 2 is an explanatory diagram illustrating a state in which oxidation and reduction of redox molecules are repeated.

FIG. 3A is an explanatory diagram illustrating a biomolecule measurement method according to Embodiment 2 of the present invention.

FIG. 3B is an explanatory diagram illustrating a biomolecule measurement method according to Embodiment 2 of the present invention.

FIG. 3C is an explanatory diagram illustrating a biomolecule measurement method according to Embodiment 2 of the present invention.

FIG. 4 is a characteristics diagram showing measurement results (dotted line) of an aqueous solution 121 containing no biomolecules 122 and measurement results (solid line) of an aqueous solution 121 containing biomolecules 122.

FIG. 5A is an explanatory diagram illustrating a biomolecule measurement method according to Embodiment 3 of the present invention.

FIG. 5B is an explanatory diagram illustrating a biomolecule measurement method according to Embodiment 3 of the present invention.

FIG. 5C is an explanatory diagram illustrating a biomolecule measurement method according to Embodiment 3 of the present invention.

FIG. 6 is a characteristics diagram showing measurement results (dotted line) of an aqueous solution 121 containing no biomolecules 122 and measurement results (solid line) of an aqueous solution 121 containing biomolecules 122.

### DETAILED DESCRIPTION OF ILLUSTRATIVE EMBODIMENTS

A biomolecule measurement device and method according to embodiments of the present invention will be described below.

### Embodiment 1

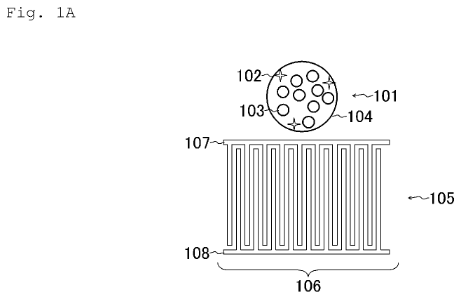

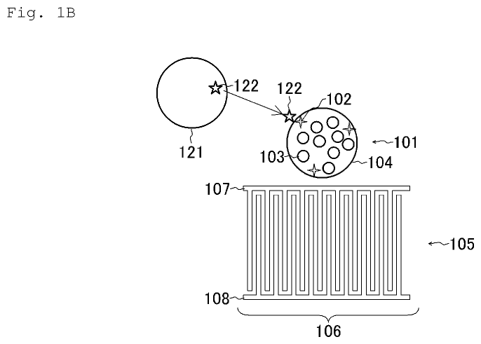

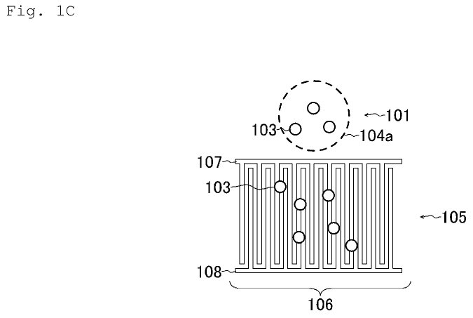

First, a biomolecule measurement method in Embodiment 1 of the present invention will be described with reference to FIG. 1A to FIG. 1C.

First, as shown in FIG. 1A, in the biomolecule measurement method, particles 101 of a sustained-release gel 104 containing enzymes 102 for measurement of target biomolecules and a plurality of detection molecules 103 are prepared (first process).

Here, when the measurement target biomolecules are, for example, glutamic acid, a glutamic acid oxidase may be the enzyme 102 that reacts with the measurement target biomolecules. The detection molecules 103 are molecules measured according to an electrochemical reaction using a measurement device (a measurement chip 105) to be described below. For example, redox molecules such as ferrocene and potassium ferricyanide can be used as the detection molecules 103.

The sustained-release gel 104 can be composed of a gel-like substance such as a reactive hydrogel which forms a sol by reacting with a product (generated molecules) produced (generated) by a reaction (enzymatic reaction) of measurement target biomolecules with the enzyme 102. For the sustained-release gel 104, for example, phenylboronic acid phenylmethoxycarbonyl (BPmoc-F₃) can be used, which is converted into a sol by hydrogen peroxide which is an oxidase product (refer to NPL 3). Here, the production of the particles 101 of the sustained-release gel 104 will be described below.

In Embodiment 1, the particles 101 are disposed on the measurement chip 105. For example, the measurement chip 105 has a measurement region 106 in which an aqueous solution containing measurement target biomolecules can come in contact with a surface of a substrate and is an electrochemical measurement device that performs electrochemical measurement using a comb electrode composed of a first electrode 107 and a second electrode 108 formed in the measurement region 106, and a reference electrode and a counter electrode (not shown).

In Embodiment 1, the detection molecules 103 are redox molecules. The measurement chip 105 measures the detection molecules 103 according to an electrochemical reaction using the first electrode 107 and the second electrode 108.

Next, as shown in FIG. 1B, in the measurement region 106 of the measurement chip 105, measurement target biomolecules 122 are brought into contact with the particles 101 (second process). In Embodiment 1, when an aqueous solution 121 containing the biomolecules 122 is supplied to the measurement region 106 of the measurement chip 105, the biomolecules 122 are brought into contact with the particles 101. The aqueous solution 121 is, for example, blood or plasma. When biomolecules come in contact with the particles 101, the biomolecules 122 react with the enzyme 102 contained in the particles 101. For example, with the enzyme 102 which is a glutamic acid oxidase, the biomolecules 122 of glutamic acid cause hydrogen peroxide to be produced as a product according to a reaction of “C₅H₉NO₄+H₂O→C₅H₆O₅+NH₃+H₂O₂”. In this manner, the sustained-release gel 104 is converted into a sol by reacting with a product produced according to the reaction of the biomolecules 122 with the enzyme 102.

Next, as shown in FIG. 1C, the detection molecules 103 are measured (third process). A plurality of contained detection molecules 103 are released from a sustained-release gel 104a which is converted into a sol in the measurement region 106 of the measurement chip 105, and the released detection molecules 103 are measured by the measurement chip 105. For example, the biomolecules 122 and the particles 101 come into contact with each other in the aqueous solution 121, and the detection molecules 103 are released from the sustained-release gel 104a into the aqueous solution 121. The detection molecules 103 released into the aqueous solution 121 can be measured using an electrochemical reaction.

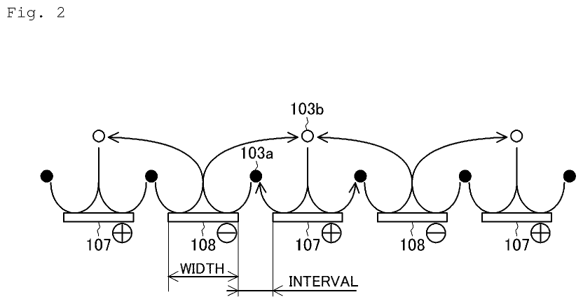

That is, the detection molecules 103 released into the aqueous solution 121 move in the aqueous solution 121 and come into contact with the first electrode 107 or the second electrode 108 of the measurement region 106. As shown in FIG. 2, the detection molecule 103 that is a redox molecule is oxidized at the first electrode 107 that is an anode to form an oxidant 103a. In addition, the oxidant 103a is reduced at the second electrode 108 that is a cathode to form a reductant 103b. The reductant 103b is oxidized at the first electrode 107 to form an oxidant 103a. In the comb electrode composed of the first electrode 107 and the second electrode 108, the above oxidation and reduction are repeated (redox cycle) between the adjacent first electrode 107 and second electrode 108, and an apparent value of a current flowing between the first electrode 107 and the second electrode 108 increases. Therefore, the detection molecules 103 can be measured from the increase or decrease in the current value.

According to Embodiment 1 described above, for example, according to the reaction of one biomolecule 122 with the enzyme 102, as a result, a plurality of detection molecules 103 are released from one particle 101. Therefore, due to the presence of one biomolecule 122, a plurality of detection molecules 103 are measured, and the detection sensitivity can increase.

In addition, regarding the measurement chip 105, it is sufficient for a structure of the above electrode composed of the first electrode 107 and the second electrode 108 to be provided, and if the particles 101 are disposed and used in a measurement region of the measurement chip during measurement, the above measurement of the biomolecules can be performed. Therefore, it is not necessary to chemically modify the measurement chip in advance, and according to Embodiment 1, the measurement can be performed without using a dedicated measurement chip having an expiration date. In addition, since the sustained-release gel 104 can be converted into a sol in a short time and the detection molecules 103 can also be measured in a short time, the biomolecules 122 can be efficiently measured in a short time according to Embodiment 1.

In addition, when the widths of the first electrode 107 and the second electrode 108 are narrower, and the interval between the first electrode 107 and the second electrode 108 is narrower, the frequency of repetition of the above oxidation and reduction per unit time is improved, and further improvement in sensitivity can be expected. Here, while a case in which the aqueous solution 121 containing the biomolecules 122 is brought into contact with the particles 101 has been exemplified in Embodiment 1, the present invention is not limited to a case using an aqueous solution. For example, when biomolecules are a gas, the biomolecules can be directly brought into contact with the particles 101 without using an aqueous solution.

Next, an example of a method of producing particles 101 of a sustained-release gel 104 containing enzymes 102 for measurement of target biomolecules and a plurality of detection molecules will be described (refer to NPL 4).

First, a first aqueous solution is prepared by mixing BPmoc-F₃, which is a material of the enzyme 102 and the sustained-release gel 104. On the other hand, a second aqueous solution is prepared by mixing in the detection molecules 103. In the second aqueous solution, the amount of the detection molecules 103 added is known.

Next, a flow path substrate including a first flow path, a second flow path, and a third flow path is prepared. The first aqueous solution is put into the first flow path, the second aqueous solution is put into the second flow path, an oil is put into the third flow path, these liquid components are propelled and merged, and thus the first aqueous solution, the second aqueous solution, and the oil are mixed, and the mixture is discharged into water.

The mixed solution discharged into water forms the particles 101 having a predetermined size corresponding to a discharge amount per unit time. The particles 101 are precipitated in water, while the oil is suspended in water, and thus the particles 101 and the oil are separated from each other. A total amount of the detection molecules 103 contained in all of the obtained particles 101 is a known amount of the detection molecules 103 added in the second aqueous solution.

Using a known addition amount of the detection molecules 103 produced in this manner, regarding the aqueous solutions 121 containing the biomolecules 122 with different concentrations, the biomolecules 122 can be measured according to the above measurement method, and a calibration curve can be created. Biomolecules can be quantitatively measured using the calibration curve and the particles 101 of the sustained-release gel 104 prepared according to the above production method.

Here, in the method of producing the particles 101 of the sustained-release gel 104 described above, a case in which the particles 101 are produced using an oil has been exemplified. However, when the first aqueous solution and the second aqueous solution are mixed and discharged without using an oil, a membrane of the sustained-release gel 104 containing the enzymes 102 and a plurality of detection molecules 103 can be formed.

### Embodiment 2

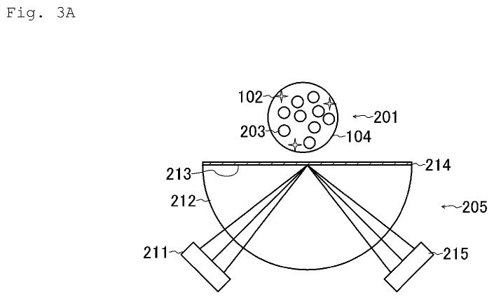

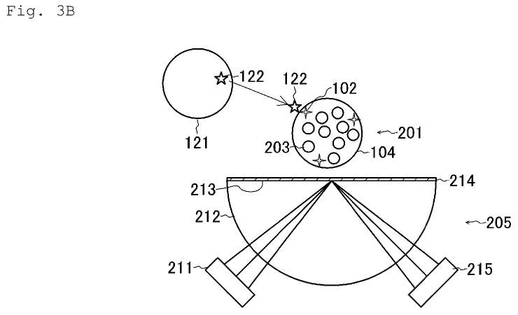

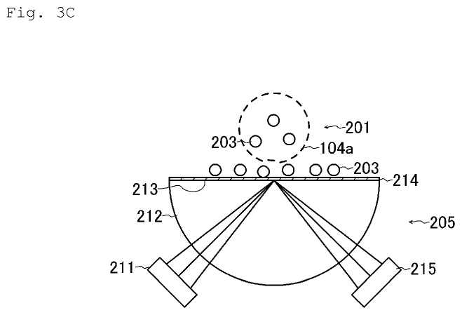

Next, a biomolecule measurement method in Embodiment 2 of the present invention will be described with reference to FIG. 3A to FIG. 3C.

First, as shown in FIG. 3A, in the biomolecule measurement method, particles 201 of a sustained-release gel 104 containing enzymes 102 for measurement of target biomolecules and a plurality of detection molecules 203 are prepared (first process). The enzyme 102 and the sustained-release gel 104 are the same as those in Embodiment 1 described above. The detection molecules 203 have a smaller molecular weight than the measurement target biomolecules. When the measurement target biomolecules are glutamic acid, for example, sugars such as glucose and fructose can be used as the detection molecules 203. The detection molecules 203 are molecules measured by a surface plasmon resonance method using a measurement device 205 to be described below.

In Embodiment 2, the particles 201 are disposed on the measurement device 205. The measurement device 205 has a measurement region with which an aqueous solution containing measurement target biomolecules can come in contact and measures the detection molecules 203 in the measurement region. The measurement device 205 is a well-known SPR device, and includes a light source 211, a measurement prism 212, a measurement surface 213, an Au layer 214, and a sensor 215. An area above the Au layer 214 is the measurement region. The Au layer 214 has a thickness of about 50 nm. The sensor 215 is composed of an imaging element such as a so-called CCD image sensor. The SPR device is, for example, a “Smart SPR SS-100” (commercially available from NTT Advanced Technology Corporation). For example, a sensor chip having an Au layer 214 formed thereon may be formed on a glass substrate such as K7, and the sensor chip may be disposed on the measurement surface 213 of the measurement prism 212.

Light emitted from the light source 211 is collected and incident on the measurement prism 212 and is emitted to the measurement surface 213 of the measurement prism 212. The light that has been transmitted through the measurement prism 212 is emitted to the back surface of the Au layer 214. The light emitted in this manner is reflected at the back surface of the Au layer 214 and is photoelectrically converted by the sensor 215 to obtain an intensity (light intensity). This light intensity (reflectance) changes according to the amount of the detection molecules 203 on the Au layer 214, and this change is detected by the sensor 215 as a change in the SPR angle. Based on the detected change, the detection molecules 203 are measured (quantified).

Next, as shown in FIG. 3B, the measurement target biomolecules 122 are brought into contact with the particles 201 on the Au layer 214 as a measurement region of the measurement device 205 (second process). In Embodiment 2, when the aqueous solution 121 containing the biomolecules 122 is supplied onto the Au layer 214 as a measurement region of the measurement device 205, the biomolecules 122 are brought into contact with the particles 201. The aqueous solution 121 and the biomolecules 122 are the same as those in Embodiment 1 described above. When the biomolecules 122 come into contact with the particles 201, the biomolecules 122 react with the enzyme 102 contained in the particles 201, and hydrogen peroxide is produced as a product. In this manner, the sustained-release gel 104 is converted into a sol by reacting with a product (hydrogen peroxide) produced by a reaction of the biomolecules 122 with the enzyme 102.

Next, as shown in FIG. 3C, the detection molecules 203 are measured (third process). A plurality of contained detection molecules 203 are released from the sustained-release gel 104a converted into a sol on the Au layer 214 as a measurement region of the measurement device 205, and approach (come in contact with) the Au layer 214, and are measured by the measurement device 205.

For example, the biomolecules 122 and the particles 201 come in contact with each other in the aqueous solution 121, and the detection molecules 203 are released into the aqueous solution 121 from the sustained-release gel 104a.

The detection molecules 203 released into the aqueous solution 121 move in the aqueous solution 121 and approach the Au layer 214, and can be measured using a known surface plasmon resonance method by the measurement device 205.

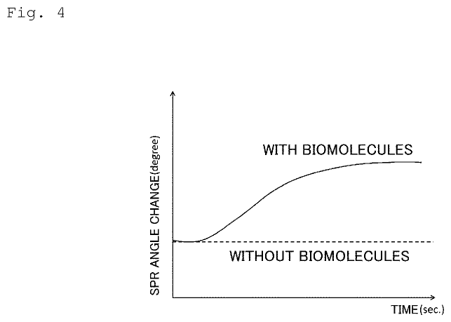

For example, as shown in FIG. 4, the particles 201 are brought into contact with the aqueous solution 121 containing no biomolecules 122, the above measurement is performed, and the measurement result is obtained as an initial value (dotted line). The initial value (dotted line) is subtracted from the actual measurement result (solid line), and thus a measurement result corresponding to the state in which the biomolecules 122 are contained can be obtained.

According to Embodiment 2 described above, for example, according to the reaction of one biomolecule 122 with the enzyme 102, as a result, a plurality of detection molecules 203 are released from one particle 201. Therefore, due to the presence of one biomolecule 122, a plurality of detection molecules 203 are measured, and the detection sensitivity can increase.

In addition, for example, a flow path may be formed on the Au layer 214, a buffer solution in which the particles 201 are dispersed is allowed to flow through the flow path, and the aqueous solution 121 is added thereto, and thus the above measurement is performed. In this manner, the particles 201 receive buoyancy in the flow path under the flow (liquid sending) condition, and are prevented from approaching the Au layer 214, and measurement of the particles 201 can be curbed.

In addition, in the measurement using the measurement device 205, the following measurement chip can be used. The measurement chip includes a second flow path through which the aqueous solution 121 can be added to the first flow path through which a buffer solution flows, and has a measurement region in the first flow path downstream from a part in which the aqueous solution 121 is added through the second flow path. When the measurement chip is used, an Au layer is formed in the measurement region of the first flow path. The measurement chip is placed on the measurement surface of the measurement device, the particles 201 are added to the buffer solution and flow through the first flow path during measurement, the aqueous solution 121 containing the biomolecules 122 is added through the second flow path, and thus the above measurement can be performed. Therefore, it is not necessary to chemically modify the measurement chip in advance, and according to Embodiment 2, the measurement can be performed without using a dedicated measurement chip having an expiration date. In addition, since the sustained-release gel 104 can be converted into a sol in a short time and the detection molecules 203 can also be measured in a short time, the biomolecules 122 can be efficiently measured in a short time according to Embodiment 2.

### Embodiment 3

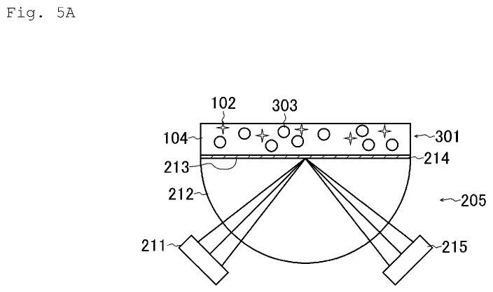

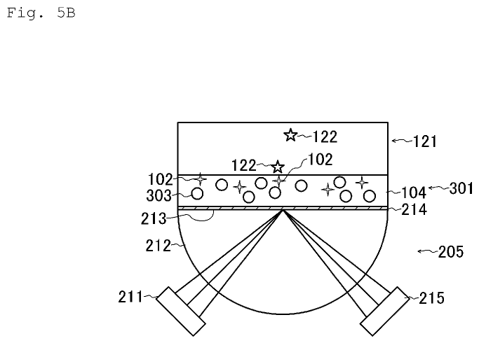

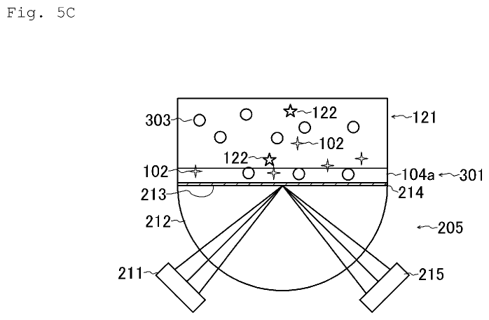

Next, a biomolecule measurement method in Embodiment 3 of the present invention will be described with reference to FIG. 5A to FIG. 5C.

First, as shown in FIG. 5A, in the biomolecule measurement method, a membrane 301 of a sustained-release gel 104 containing enzymes 102 for measurement of target biomolecules and a plurality of detection molecules 303 is prepared (first process). The enzyme 102 and the sustained-release gel 104 are the same as those in Embodiments 1 and 2 described above. The detection molecules 303 have a larger molecular weight than the measurement target biomolecules. When the measurement target biomolecules are glutamic acid, for example, a polysaccharide such as dextran can be used as the detection molecules 303. The detection molecules 303 are molecules measured by a surface plasmon resonance method using the measurement device 205.

In Embodiment 3, the membrane 301 is disposed on the measurement device 205. The measurement device 205 is the same as those in Embodiment 2 described above. In Embodiment 3, the change in the thickness of the membrane 301 containing the detection molecules 303 is measured by the measurement device 205. For example, a measurement chip having an Au layer 214 formed thereon is formed on a glass substrate such as K7. When the measurement chip is disposed on the measurement surface 213 of the measurement prism 212, the Au layer 214 is disposed on the measurement surface 213 via a glass substrate of the measurement chip. The membrane 301 is disposed on the Au layer 214 of the measurement chip.

Next, as shown in FIG. 5B, the measurement target biomolecules 122 are brought into contact with the membrane 301 on the Au layer 214 as a measurement region (second process). In Embodiment 3, when the aqueous solution 121 containing the biomolecules 122 is supplied onto the Au layer 214 as a measurement region, the biomolecules 122 are brought into contact with the membrane 301. The aqueous solution 121 and the biomolecules 122 are the same as those in Embodiments 1 and 2 described above. When biomolecules come in contact with the membrane 301, the biomolecules 122 react with the enzyme 102 containing the membrane 301, and hydrogen peroxide is produced as a product. In this manner, the sustained-release gel 104 is converted into a sol by reacting with a product produced by a reaction of the biomolecules 122 with the enzyme 102.

Next, as shown in FIG. 5C, the change in the thickness of the membrane 301 is measured (third process). A plurality of contained detection molecules 303 are released from the sustained-release gel 104a converted into a sol. For example, the biomolecules 122 and the membrane 301 come into contact with each other in the aqueous solution 121, and the detection molecules 303 are released from the sustained-release gel 104a converted into a sol into the aqueous solution 121. When the detection molecules 303 are released from the membrane 301, the amount of the detection molecules 303 in the membrane 301 in contact with the Au layer 214 decreases and the membrane 301 becomes thin. The change in the refractive index (SPR angle change) decreases in response to the decrease the thickness of the membrane 301 (decrease in the detection molecules 303 in the membrane 301), and this decrease is measured by the measurement chip 105.

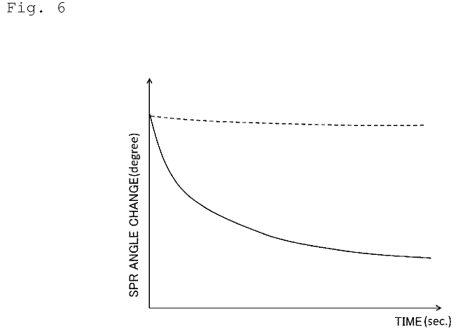

As shown in FIG. 6, when the membrane 301 is brought into contact with the aqueous solution 121 containing no biomolecules 122 and the above measurement is performed, the change in the SPR angle is not measured (dotted line). On the other hand, when the membrane 301 is brought into contact with the aqueous solution 121 containing the biomolecules 122 and the above measurement is performed, the decrease in the change of the SPR angle is measured (solid line).

According to Embodiment 3 described above, for example, according to the reaction of one biomolecule 122 with the enzyme 102, as a result, a plurality of detection molecules 303 are released from one membrane 301, and the membrane 301 becomes thin. Therefore, due to the presence of one biomolecule 122, the decrease in the thickness of the membrane 301 resulting from the decrease in the number of the plurality of detection molecules 303 from the membrane 301 is measured, and the detection sensitivity can increase.

In addition, for example, using a measurement chip including one flow path, the membrane 301 is formed at a part of the Au layer 214 in the flow path, and the aqueous solution 121 flows through the flow path, and thus the above measurement is performed. As described above, since the measurement can be performed using a measurement chip having a simple structure, it is possible to minimize the increase in the size of the device. In addition, since the detection molecules 303 are contained in the sustained-release gel 104, moisture retention is secured and the functionality of the detection molecules 303 is easily maintained for a longer time. In addition, since the sustained-release gel 104 can be converted into a sol in a short time and the decrease in the thickness of the membrane 301 due to release of the plurality of detection molecules 303 can also be measured in a short time, the biomolecules 122 can be efficiently measured in a short time according to Embodiment 3.

As described above, according to embodiments of the present invention, detection molecules measured by the measurement device and enzymes for measurement of target biomolecules are contained in a sustained-release gel which is converted into a sol by reacting with a product produced by a reaction of the biomolecules with the enzyme. Therefore, according to the present invention, biomolecules can be measured efficiently in a short time with high detection sensitivity without using a dedicated measurement chip having an expiration date. The present invention can be applied for biochemical tests such as a blood component test, body fluid analysis, and odor component analysis. According to embodiments of the present invention, these analyses can be performed with sensitivity without providing a special concentration mechanism in a collection mechanism of a measurement target object.

Here, it is apparent that the present invention is not limited to the embodiments described above, and many modifications and combinations can be implemented by those skilled in the art within the technical scope of the present invention.

### REFERENCE SIGNS LIST

101 Particle

102 Enzyme

103 Detection molecule

103a Oxidant

103b Reductant

104, 104a Sustained-release gel

105 Measurement chip (measurement device)

106 Measurement region

107 First electrode

108 Second electrode

121 Aqueous solution

122 Biomolecule.

In the claims:

1.-8. (canceled)

9. A biomolecule measurement device, comprising:
a measurement device having a measurement region in which detection molecules are measured; and
particles of a sustained-release gel comprising an enzyme for measurement of target biomolecules and a plurality of the detection molecules, the sustained-release gel configured to be converted into a sol by reacting with a product produced by a reaction of the target biomolecules with the enzyme and release the detection molecules.

10. The biomolecule measurement device according to claim 9, wherein:
the detection molecules are redox molecules; and
the measurement device includes a first electrode and a second electrode disposed in the measurement region and configured to measure the detection molecules according to an electrochemical reaction.

11. The biomolecule measurement device according to claim 9, wherein:
the detection molecules have a smaller molecular weight than the biomolecules; and
the measurement device is configured to measure the detection molecules by a surface plasmon resonance method.

12. The biomolecule measurement device according to claim 9, wherein the sustained-release gel is phenylboronic acid phenylmethoxycarbonyl (BPmoc-F₃).

13. The biomolecule measurement device according to claim 9, wherein the enzyme is a glutamic acid oxidase, the target biomolecules are glutamic acid, and the detection molecules are hydrogen peroxide.

14. A biomolecule measurement device, comprising:
a membrane of a sustained-release gel comprising an enzyme for measurement of target biomolecules and a plurality of the detection molecules, the sustained-release gel configured to be converted into a sol by reacting with a product produced by a reaction of the biomolecules with the enzyme and release the detection molecules; and
a measurement device configured to measure a change in the thickness of the membrane by a surface plasmon resonance method, wherein the detection molecules have a larger molecular weight than the biomolecules.

15. A biomolecule measurement method, comprising:
a first process in which particles of a sustained-release gel are prepared, the sustained-release gel comprising an enzyme for measurement of target biomolecules and a plurality of the detection molecules, and the sustained-release gel being configured to be converted into a sol by reacting with a product produced by a reaction of the target biomolecules with the enzyme and release the detection molecules;
a second process in which the biomolecules are brought into contact with the particles; and
a third process in which, after the biomolecules are brought into contact with the particles, the detection molecules are measured.

16. The biomolecule measurement method according to claim 15, wherein:
the detection molecules are redox molecules.

17. The biomolecule measurement method according to claim 16, wherein in the third process, the detection molecules are measured according to an electrochemical reaction.

18. The biomolecule measurement method according to claim 15, wherein the detection molecules have a smaller molecular weight than the biomolecules.

19. The biomolecule measurement method according to claim 18, wherein, in the third process, the detection molecules are measured by a surface plasmon resonance method.
